(19) United States
(12) Patent Application Publication
(10) Pub. No.: US 2025/0380921 A1
(43) Pub. Date: Dec. 18, 2025

(54) BONE DENSITY MEASUREMENTS BASED ON COMPUTED TOMOGRAPHY IMAGES

(71) Applicant: Lundquist Institute for Biomedical Innovation at Harbor-UCLA Medical Center, Torrance, CA (US)

(72) Inventors: Matthew J. BUDOFF, Torrance, CA (US); Dong LI, Torrance, CA (US); Songshou MAO, Torrance, CA (US); Christopher DAILING, Torrance, CA (US)

(21) Appl. No.: 19/315,205

(22) Filed: Aug. 29, 2025

Related U.S. Application Data
(63) Continuation of application No. PCT/IB2024/052231, filed on Mar. 7, 2024.
(60) Provisional application No. 63/492,846, filed on Mar. 29, 2023.

(51) Int. Cl. A61B 6/50; A61B 6/00; A61B 6/03; A61B 6/58; G06T 7/00; G06T 7/66; G06V 10/25; G06V 10/26; G16H 50/30

(52) U.S. Cl. CPC A61B 6/505; A61B 6/032; A61B 6/5217; A61B 6/563; A61B 6/582; G06T 7/0012; G06T 7/66; G06V 10/25; G06V 10/26; G16H 50/30; G06T 2207/10081; G06T 2207/30012; G06V 2201/033

(57) ABSTRACT

CT images from the neck, lung, cardiac, abdominal, pelvis, hip, spine or lower extremity areas are obtained and analyzed by a computer to measure trabecular bone density on each level it is available, in the spine and hip. Any bone tissue associated with vertebrae that is fractured is excluded, along with Schmorl's nodes, large blood vessels, and cortical bone, to accurately average the trabecular bone density on a scan. The density of fat, heart, and muscle tissue of a subject is used to calibrate the results. That data may be input to an absolute fracture risk model (Fracture Risk Algorithm calculator). Information such as bone density, T score, Z score, fracture risk, and identification of vertebral fractures (if present) may be determined and reported.

### 1. CROSS-REFERENCE

This application is a continuation of PCT/IB2024/052231, filed Mar. 7, 2024, which claims the benefit of U.S. Provisional Patent Application No. 63/492,846, filed Mar. 29, 2023, said applications are incorporated herein by reference in their entirety for all purposes.

### BACKGROUND

### 1. Field

The present disclosure relates to the field of bone density measurement.

### 2. Description of the Related Art

Osteoporosis is the most common metabolic disorder of bone. Bone strength is dependent on its density and therefore the measurement of bone density is important to predict osteoporosis-related fractures. The femoral neck and spine are typically the most sensitive sites and usually the first site to experience osteoporosis fractures. Computed tomography (CT) is an imaging technique used for three-dimensional bone density measurement.

### SUMMARY

The systems and methods described herein may be used to help diagnose and predict early osteoporosis and bone fractures using (e.g., CT) images and/or other information, and/or may have other uses, for example. These systems and methods are configured to make bone density determinations based on a large collection of CT slices (e.g., of all of the vertebrae in the subject and more), which is advantageous relative to prior systems, because prior systems only utilized certain images from selected structures for bone density determinations. In addition, individual patient specific calibration is performed using known densities of body tissue naturally included in a scan (e.g., fat, heart tissue, muscle tissue, etc.), making the present systems and methods more accurate than prior phantomless techniques, and more convenient than techniques that require a separate dedicated bone density phantom below a subject during a scan. Also, the present systems and methods comprise an automated system configured to measure trabecular bone density without including large blood vessels, Schmorl's nodes, and vertebral fractures in the analysis, which enhances accuracy of bone density determinations.

The following is a non-exhaustive listing of some aspects of the present techniques. These and other aspects are described in the following disclosure.

Some aspects include a method comprising obtaining one or more images of a spine region comprising thoracic and/or lumbar vertebrae, and/or a hip, by computed tomography (CT); identifying a spinal column automatically based on the one or more images; and determining a bone density measurement based on the one or more images and the identification of the spinal column.

In some embodiments, the one or more images comprise CT images including a lumbar and/or thoracic spine.

In some embodiments, the method comprises automatically detecting thoracic and/or lumbar spinal vertebrae present in the one or more images and segmenting individual vertebrae.

In some embodiments, the method comprises quantifying a height of each spinal vertebrae, and labelling each spinal vertebrae as thoracic or lumbar.

In some embodiments, the method comprises determining vertebral fractures for any vertebrae with a height loss more than 20% of an average vertebral height.

In some embodiments, the method comprises excluding bone from any vertebrae determined to be fractured from the bone density measurement.

In some embodiments, the method comprises identifying trabecular bone for each non-fractured vertebrae in the one or more images by identifying a center of mass for each non-fractured vertebra in the one or more images.

In some embodiments, the method comprises defining a region of interest (ROI) to include only trabecular bone in the one or more images.

In some embodiments, the one or more images each comprise an axial CT slice.

In some embodiments, the method comprises maximizing a size of the ROI for each axial CT slice, while avoiding cortical bone, bone islands, vertebral fractures, areas with large vessels, and/or calcified herniated discs based upon Hounsfield units (HU) associated with CT.

In some embodiments, the method comprises determining a mean density of the trabecular bone on every available axial CT slice in the one or more images.

In some embodiments, the method comprises averaging the mean density from each slice to obtain a bone mineral density (BMD) measurement.

In some embodiments, the method comprises using a subject's heart density, chest mass, and/or chest wall/abdominal or hip fat to calibrate the patient, scanner, and/or scan parameters used to determine the bone density measurement.

In some embodiments, the method comprises using a subject's heart, fat, and/or muscle (in the chest wall, abdominal wall, and/or hip area) to calibrate vertebral and/or femoral bone density measures (e.g., from CTHU to mg/cc in CaHA).

In some embodiments, calibrating comprises converting HU associated with CT to Mg/cc of calcium hydroxyapatite (CaHA).

In some embodiments, the method comprises determining a T score and a Z score for thoracic, lumbar, both thoracic and lumbar spines, and/or a hip, depending on what is included in the one or more images.

In some embodiments, the method comprises determining a Schmorl's node size, depth, and/or quantity.

In some embodiments, the method comprises determining a FRAX score based on the Schmorl's node size, depth, location and/or quantity; a BMD for the thoracic and/or lumbar spines, and/or a hip; and/or a whole body BMD.

In some embodiments, the bone density measurement comprises a whole body BMD measurement automatically determined by a computing system.

Some aspects include a computer program product comprising a non-transitory computer readable medium having instructions recorded thereon. The instructions, when executed by a computer, implement some or all of the operations in the above mentioned methods.

Some aspects include a system, including an imager, one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 provides a schematic illustration of a system configured for determining a bone density measurement.

FIG. 2 illustrates thoracic, lumbar spine, and hip neck density measurement images.

FIG. 3 demonstrates a representative calibration based on fat and heart tissue density.

FIG. 4 illustrates the use of chest wall/abdominal or hip fat, muscle tissue, and heart tissue as a calibrating phantom for CT scanner, patient, and/or CT scan parameters. FIG. 4 illustrates using a subject's heart, fat, and muscle (in the chest wall, abdominal wall, and hip area) as a calibrating factor to calibrate vertebral or femoral bone density measures.

FIG. 5 illustrates trabecular bone in different CT scan images, and assessing scan heights from coronal, sagittal and axial views.

FIG. 6 is a diagram that illustrates an exemplary computing device.

FIG. 7 illustrates a method for determining a bone density measurement.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF CERTAIN EMBODIMENTS

To mitigate the problems described herein, the inventor had to both invent solutions and, in some cases just as importantly, recognize problems overlooked (or not yet foreseen) by others in the field of bone density measurement. The inventor(s) wish(es) to emphasize the difficulty of recognizing those problems that are nascent and will become much more apparent in the future should trends in industry continue as the inventor expects. Further, because multiple problems are addressed, it should be understood that some embodiments are problem-specific, and not all embodiments address every problem with traditional systems described herein or provide every benefit described herein. That said, improvements that solve various permutations of these problems are described below.

For example, measuring bone density using CT without a phantom below a subject (rarely done in clinical practice) is challenging. Adjusting the brightness (as one example parameter) on a (CT) scan to account for all of the different possible variables associated with a scan requires a calibration technique that typically includes a phantom. Given the large number of CT scanners, and even larger numbers of scan techniques and parameters that can be used, calibration is critical. The systems and methods described below facilitate individual patient specific calibration using known densities of body tissue naturally included in a scan (e.g., fat, heart tissue, muscle tissue, etc.), making the present systems and methods more accurate than prior phantomless techniques, and more convenient than techniques that require a separate dedicated bone density phantom below a subject during a scan.

Further, large blood vessels, Schmorl's nodes, and vertebral fractures can affect the measured density of trabecular bone if included in a measurement analysis. The present systems and methods comprise an automated system configured to measure trabecular bone density without including these other structures in the analysis, which enhances accuracy of bone density determinations.

In addition, automated detection of vertebral fractures is rarely performed in clinical practice, as it is time consuming and still requires manual measurements. The present systems and methods are configured to automate the detection of vertebral fractures and add that clinical information to a scan with high accuracy. The present systems and methods are configured to assess the average vertebral scan height more accurately than manual measurement, and compare vertebral scan heights to the height of an average vertebrae, and determine that a vertebrae is fractured if the height is less than a threshold amount less than average.

Advantageously, with the present systems and methods, CT images from the neck, lung, cardiac, abdominal, pelvis, hip, spine or lower extremity areas are obtained and analyzed by a computer to measure trabecular bone density on each level it is available, in the spine and hip and/or in other locations. Any bone tissue associated with vertebrae that is fractured is excluded, along with Schmorl's nodes, large blood vessels, and cortical bone, to accurately average the trabecular bone density on a scan. The density of fat, heart, and muscle tissue of a subject is used to calibrate the results. That data may be input to an absolute fracture risk model (Fracture Risk Algorithm calculator). Information such as bone density, T score, Z score, fracture risk, and identification of vertebral fractures (if present) may be determined and reported.

Until now, there had been no phantomless bone density measurement technique that makes density corrections based on known subject specific tissue (e.g., fat, heart, muscle, etc.) densities. Until now there had been no automated vertebral detection incorporated with bone density measurements. Until now, uniform T and Z score determinations were not developed and validated based on extensive population based studies. Until now, there had been no technique that systematically excluded Schmorl's nodes, vertebral fractures, large blood vessels, and cortical bone, from bone density analyses, which now facilitates a more accurate assessment of the density of trabecular bone in a subject.

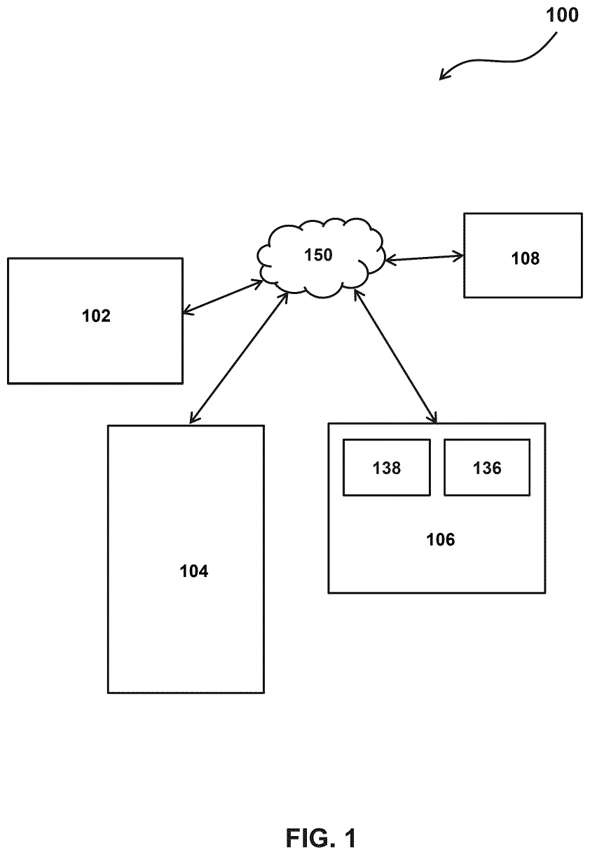

FIG. 1 provides a schematic illustration of a system 100 configured for determining a bone density measurement. System 100 comprises an imager 102, one or more processors 104, one or more computing devices 106, external resources 108, a network 150, and/or other components. Each of these components is described in turn below.

Imager 102 is configured to obtain images of a subject. The images may be of a spine region comprising thoracic and/or lumbar vertebrae, and/or a hip, and/or other regions on the subject. These may include images from a subject's neck, lung, cardiac, abdominal, pelvis, hip, spine and/or lower extremity regions, for example. In some embodiments, imager 102 comprises any device capable of generating such images. Suitable devices may include, but are not limited to, an electron beam tomography (EBT) scanner provided by GE or other CT scanners provided by GE, Siemens, Toshiba and Philips and other manufacturers.

For example, imager 102 may be or include a CT scanner. A CT scanner is configured to aggregate multiple X-ray images of a subject taken in series from different angles around the subject's body. A CT scanner and/or one or more processors such as processor(s) 104 associated with the CT scanner generate cross-sectional images (slices) of the various structures (e.g., bones, blood and soft tissues) inside the subject's body.

Imager 102 may be an X-ray or electron CT device including a frame, a gantry unit, and/or other components. The gantry unit may be configured to acquire projection data associated with the subject. The gantry unit may include an X-ray tube, X-ray detector, and/or other components. The X-ray tube and X-ray detector may be mounted on a ring-shaped rotating frame which is rotated by a driving unit. For an imaging operation, the subject is placed on top of a support unit and is typically inserted into an open portion of imager 102 so that the X-ray tube and detector can rotate around the subject to obtain one or more images. Imager 102 may convert a signal output from the X-ray detector into a digital signal used to generate the one or more images.

One or more processors 104 are configured to provide information processing capabilities in system 100. As such, processor(s) 104 may include one or more of a digital processor, an analog processor, a digital circuit designed to process information, an analog circuit designed to process information, a state machine, and/or other mechanisms for electronically processing information. In some embodiments, a processor 104 may be included in and/or otherwise operatively coupled with imager 102, computing device 106, and/or other components of system 100. Although one or more processors 104 are shown in FIG. 1 as a single entity, this is for illustrative purposes only. In some implementations, processor(s) 104 may include a plurality of processing units. These processing units may be physically located within the same device (e.g., imager 102, computing device 106, a server not shown in FIG. 1, etc.), or processor(s) 104 may represent processing functionality of a plurality of devices operating in coordination (e.g., a processor located within imager 102 and a second processor located within computing device 106). Processor(s) 104 may be configured to execute one or more computer program components. Processor(s) 104 may be configured to execute the computer program component by software; hardware; firmware; some combination of software, hardware, and/or firmware; and/or other mechanisms for configuring processing capabilities on processor(s) 104.

Processor(s) 104 are configured to receive one or more images of a spine region. The one or more images show thoracic and/or lumbar vertebrae, and/or a hip. The one or more images may be (previously) obtained by an imager (e.g., imager 102 described above). The imager may be configured for computed tomography (CT) and/or other imaging techniques. In some embodiments, the one or more images may comprise CT images including a lumbar and/or thoracic spine and/or other images. For example, the one or more images may each comprise an axial CT slice. The axial CT slices may cover all vertebrae in the subject, for example, and/or other structures. Making bone density determinations based on a large collection of CT slices (e.g., of all of the vertebrae in the subject and more) may be advantageous relative to prior systems, which only utilized certain images from selected structures for bone density determinations. Use of multiple vertebrae (from a large collection of CT slices) increases accuracy, allows for exclusion of vertebrae that may be fractured or have Schmorl's nodes, large veins, and/or other findings that diminish accuracy of trabecular bone related determinations. As one representative example, use of multiple vertebrae (from a large collection of CT slices) can decrease the precision error in a pre-measurement from about 2.5% to about 1.2%-1.5% (e.g., around a 50% decrease in precision error).

Processor(s) 104 are configured to automatically identify a spinal column in the one or more images. This may include automatically detecting thoracic and/or lumbar spinal vertebrae present in the one or more images and segmenting individual vertebrae, for example. Each spinal vertebrae may be labeled (e.g., by processor(s) 104 and/or other components) as thoracic or lumbar, and/or may be labelled with other information.

In some embodiments, a height (e.g., a distance from one side to another side of a vertebrae in an image) of each spinal vertebrae may be quantified (e.g., using image analysis techniques). Vertebral fractures and/or other information may be determined based on these heights. For example, vertebral fractures may be determined for any vertebrae with a height loss more than some threshold amount. In some embodiments, vertebral fractures may be determined for any vertebrae with a height loss more than some threshold amount of an average vertebral height. In some embodiments, vertebral fractures may be determined for any vertebrae with a height loss more than 10%, 15%, 20%, 30%, 40%, or 50% of an average posterior vertebral height, and/or compared to an adjacent vertebrae without vertebral fracture. In some embodiments, vertebral fractures may be determined for any vertebrae with a height loss more than 20% of an average posterior vertebral height or compared to adjacent vertebrae. For example, one or more processors 104 may be configured to automatically detect a vertebrae in an image, determine a height distance from one side of the vertebrae to another, compare that distance to an average for that distance, and determine whether a fracture is present based on that comparison (e.g., if the height distance is more than 20% less than average).

In some embodiments, processor(s) 104 are configured to detect trabecular bone for each non-fractured vertebrae in the one or more images by identifying a center of mass for each non-fractured vertebra in the one or more images. The trabecular bone is located at or near the center of the vertebrae, so measuring this from the center (of mass) towards the periphery (of the vertebrae facilitates complete imaging of the vertebrae and only includes trabecular bone, which is the bone used to calculate density from vertebrae, the hip, and/or other locations as described herein.

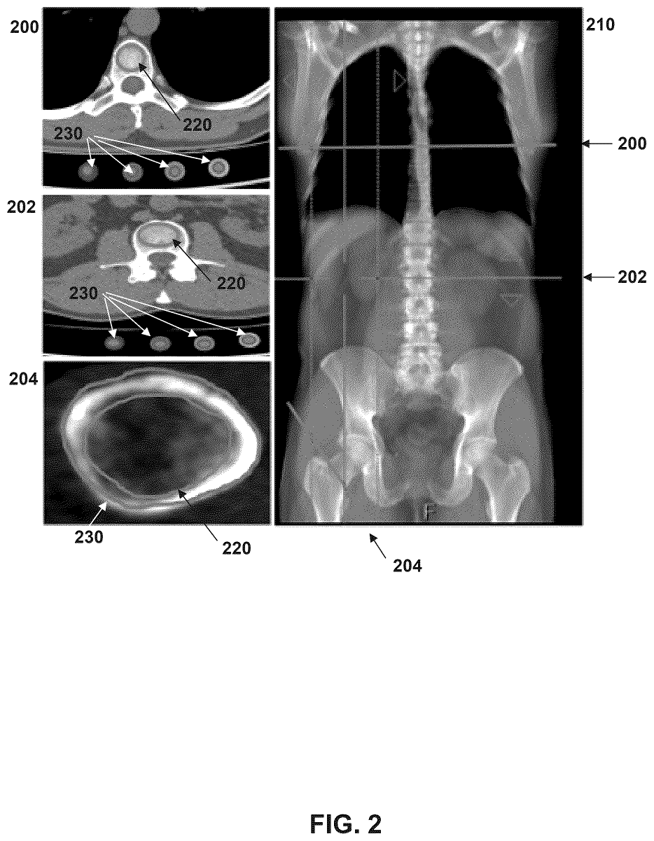

For example, FIG. 2 illustrates thoracic 200, lumbar spine 202, and hip neck 204 density measurement (CT) images. FIG. 2 also includes image 210, which shows cross sectional marking lines (labeled with corresponding image numbers) where thoracic 200, lumbar spine 202, and hip neck 204 images are obtained. In FIG. 2, circles 220 indicate the trabecular bone, and circles 230 indicate the cortical bone, which is excluded from density measurements, as described herein.

Returning to FIG. 1, processor(s) 104 may be configured to define a region of interest (ROI) to include only trabecular bone in the one or more images. The size of the ROI may be maximized for each axial CT slice, while avoiding cortical bone, bone islands, vertebral fractures, areas with large vessels, and/or calcified herniated discs. These structures may be identified (and avoided) based upon Hounsfield units (HU) associated with CT, for example. Radiologists use Hounsfield units (HU), which are used as a quantitative assessment of density, to interpret CT images. More dense tissue appears bright in a CT image and less dense tissue appears darker. A ROI may be defined based on relative light and dark portions of a CT image, for example, that correspond to different types of tissue. Processor(s) 104 may be configured to measure density from the center of the bone on each CT scan slice (e.g., vertebrae and/or hip) and stop when significant density changes occur, allowing for exclusion of cortical bone, Schmorl's node, large arteries and/or veins, vertebral fractures, and/or other areas as described herein.

Processor(s) 104 are configured to determine a bone density measurement. The bone density measurement is determined based on the one or more images and the identification of the spinal column, the center of mass determinations, ROI determinations, and/or other information. Bone from any vertebrae determined to be fractured may be excluded from the bone density measurement. In some embodiments, the bone density measurement comprises a whole body BMD measurement automatically determined by a computing system such as computing device 106 that includes processor(s) 104.

In some embodiments, the bone density measurement includes determining a mean density of the trabecular bone on every available axial CT slice in the one or more images. The bone density measurement may (also) include averaging the mean density from each slice to obtain a bone mineral density (BMD) measurement.

Processor(s) 104 are configured to use a subject's heart density, chest wall/abdominal and/or hip muscle mass and fat, and/or other tissues with known tissue densities to calibrate patient, scanner, and scan parameters used to determine the bone density measurement. Calibrating may comprise converting HU associated with CT to Mg/cc of CaHA. In some embodiments, based on the principle of X-Ray attenuation, there may be a linear association with a same slope between the density and attenuation coefficient in any organ or objects in given scan (e.g., CT image or images). Using this information, the heart, spleen, chest fat, chest muscle, etc., can be used to derive a CaHA value (e.g., concentration of CaHA in 0, 50, 100 and 200 mg/cc) and/or other information. These factors and/or other information can be used to calibrate the bone density. In some embodiments, one or more combinations of these tissues may be used for calibration. For example, chest fat+heart, or chest fat+muscle can be used to calibrate various vertebrae and/or other bone. Even if a scan does not include the chest, the abdominal and femoral fat and muscle can be used for the lumbar and femoral bone calibration respectively.

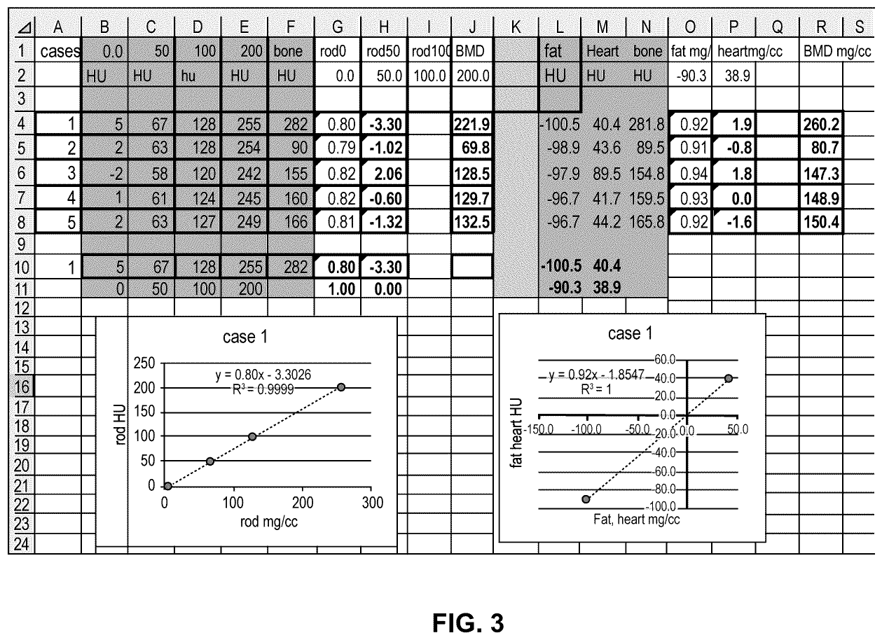

By way of a non-limiting example, FIG. 3 demonstrates a representative calibration based on fat and heart tissue density. A regression analysis is used in this example. Fat and heart tissue density may be obtained and converted from HU to mg/cc of CaHA (−90.3 and 38.9)—see column O and P and Row 2 in FIG. 3. Knowing the CT HU of the fat and heart tissue, also see column L and M in Row2 (−100.5 and 40.4 in case 1), the slope and intercept can be derived, which are 0.92 and 1.9 of column O and P in Row 4. The BMD can be derived from Bone HU-N4 to mg/cc-see column R and Row 4, =bone HU×slope+intercept. This analysis may be done automatically, and this formula may be stored on a computer system as described herein.

Returning to FIG. 1, making bone density determinations based on a large collection of CT slices (e.g., of all of the vertebrae in the subject and more); identifying an excluding fractured vertebrae; measuring trabecular bone density without including large blood vessels, Schmorl's nodes, and vertebral fractures in the analysis; using individual (average) mean density measurements; and using patient specific calibration using known densities of body tissue naturally included in a scan (e.g., fat, heart tissue, muscle tissue, etc.); are all advantageous relative to prior systems, which only utilized certain images from selected structures for bone density determinations. In some embodiments, the density is averaged over the area of trabecular bone measured on each slice of the CT scan. Vertebral bone may be visualized on hundreds of slices of data from a single CT scan. This makes the present systems and methods more accurate than prior phantomless techniques, and more convenient than techniques that require a separate dedicated bone density phantom below a subject during a scan.

In some embodiments, processor(s) 104 are configured to determine a T score, a Z score, and/or other metrics for thoracic, lumbar, both thoracic and lumbar spines, and/or a hip, depending on what is included in the one or more images. The T and/or Z scores may be determined on the density measurements and/or other information. A T score is a number that is indicative of a condition of a subject's bones relative to healthy bones of a young person. A T score indicates a difference between the subject's bone (mineral) density and that of the healthy young person. A Z score is a number that is indicative of a condition of a subject's bones relative to the bones of an average person of the subject's age. A Z score indicates a difference between the subject's bone (mineral) density and that of the average person of the subject's age. This may establish uniform reference values for calculating T and Z scores in the central bone (thoracic, lumbar, and femoral bone) and/or have other advantages.

In some embodiments, processor(s) 104 are configured to determine a Schmorl's node size, depth, location, and/or quantity. A Schmorl's node is a spinal disc herniation in which the soft tissue of the intervertebral disc bulges out into the adjacent vertebrae through an endplate defect. The Schmorl's nodes presence makes vertebral fracture more likely, and may be incorporated in the new FRAX-CT measures (described herein). Further, these nodes must be excluded from measure of bone density or they will falsely elevate a BMD measure.

Currently, there is no method to calculate a FRAX measurement using CT, only using DXA scanning. The present systems and methods provide a unique FRAX-CT score based on clinical parameters, presence of Schmorl's nodes, vertebral fractures and calibrated bone density measured the CT score; a BMD for the thoracic and/or lumbar spines, and/or a hip; and/or a whole body BMD. A FRAX-CT score indicates the subject's chances of breaking bones over the next ten years. There is no FRAX-CT score yet created for use with CT images. This is the first such FRAX-CT score with ability of CT based images to predict fracture risk.

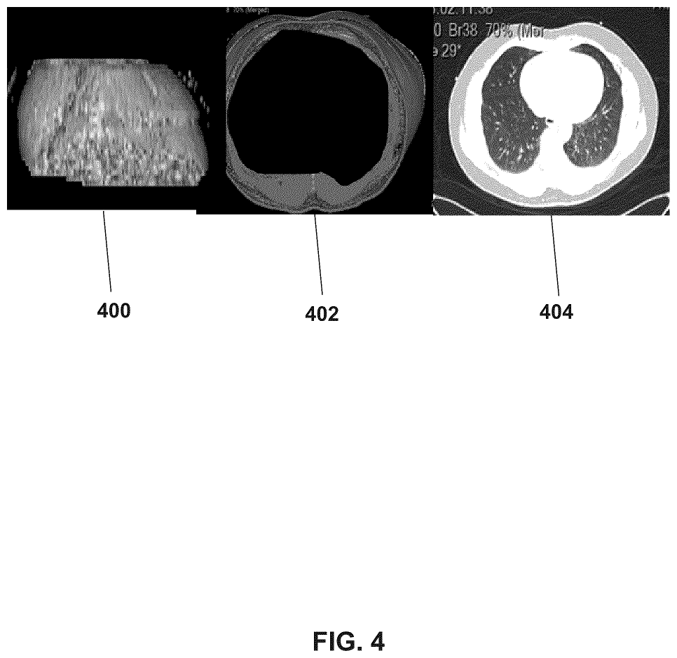

FIG. 4 illustrates the use of the use of heart 400, muscle tissue 402, and/or chest fat 404 (as three examples) as a calibrating phantom for scanner (e.g., imager 102 shown in FIG. 1), patient, scan, and/or other parameters. FIG. 4 illustrates images displayed by a computing device (such as computing device 106 shown in FIG. 1). Calibration may be performed based on such images. As described above, processor(s) 104 may use configured to use heart 400, muscle tissue 402, and/or chest fat 404 and/or other tissues with known tissue densities to calibrate patient, scanner, and scan parameters used to determine the bone density measurement. Calibrating may comprise converting HU associated with CT to Mg/cc of CaHA, for example (again as described above). This allows for more accurate assessment of bone density, by developing a calibration approach for the BMD measures (without an external phantom). Using multiple objects on each individual's CT will allow the calibration results to increase accuracy and decrease the precision error.

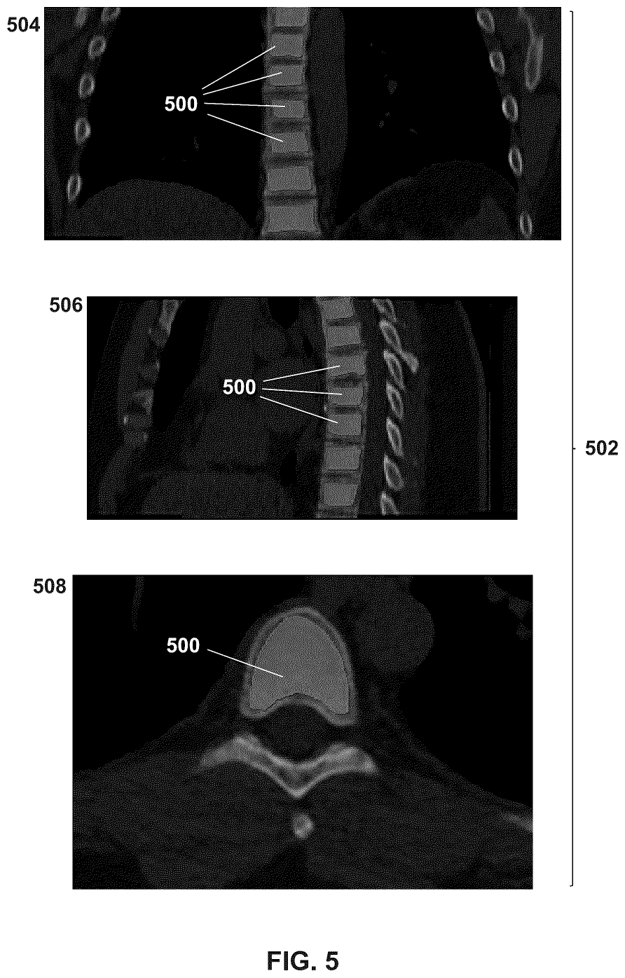

FIG. 5 illustrates examples of trabecular bone 500 in different CT scan images 502, and assessing scan heights from coronal 504, sagittal 506, and axial 508 views. Assessing scan heights comprises measurement of each vertebrae automatically and comparing the posterior vertebral height to the average of other vertebrae and adjacent vertebrae. Axial, sagittal, and coronal images are used as representative examples. Each image may be displayed by a computing device (such as computing device 106 shown in FIG. 1), a computing device included in imager 102 (FIG. 1), and/or other computing devices, for example. Hounsfield units (HU) and mg/cm3 can be displayed automatically by the computing device. From these and/or other similar images, bone density (e.g., BMD) measurements may be made, as described above. By identifying vertebral fractures automatically, these specific vertebrae can be excluded from bone density measurement, making the resulting BMD measurement more accurate, as vertebral fractures may falsely elevate the bone density.

As described above, these and/or similar CT (and/or other) images from a subject's neck, lung, cardiac, abdominal, pelvis, hip, spine and/or lower extremity regions may be obtained and provided to processor(s) 104 (FIG. 1) to measure trabecular bone density on each level it is available, in the spine, hip, and/or other regions of the subject. Any vertebrae that is fractured is excluded, along with Schmorl's nodes, large blood vessels, cortical bone, etc., to accurately average the trabecular bone density on the scan (in Hounsfield Units). The density of the subject's fat tissue, heart tissue, muscle tissue, and/or other tissues is used to calibrate the results, translating into mg/dl (bone density).

This data may be input into an absolute fracture risk model (Fracture Risk Algorithm calculator—see FRAX-CT discussion above) that takes into account age, gender, prior fracture, vertebral or hip bone density, low body mass index, rheumatoid arthritis, secondary causes of osteoporosis (type 1 (insulin dependent) diabetes, osteogenesis imperfecta in adults, untreated long-standing hyperthyroidism, hypogonadism or premature menopause (<40 years), chronic malnutrition or malabsorption, chronic liver disease, etc.), prior osteoporotic fractures (including clinical and asymptomatic vertebral fractures), parental history of hip fracture, current smoking and alcohol intake (e.g., three or more drinks/day), and/or other factors for the subject. Processor(s) 104 and/or computing device 106 may be configured to output bone density, a T Score (bone density compared to a 30 year old), a Z Score (density compared to the BMD of an age-, sex-, and ethnicity-matched reference population), a fracture risk (a FRAX-CT score), an indication of vertebral fractures (if present), and/or other information. These outputs may be used by a physician to guide diagnosis and/or treatment for the subject, and/or may have other uses.

Returning to FIG. 1, in some embodiments, one or more processors 104 are configured to execute some or all of the operations described herein based on program code stored as machine readable instructions. The machine-readable instructions may comprise instructions for obtaining images, determining bone density, and/or other operations. In some embodiments, one or more processors 104 are configured to execute one or more lines of computer code that define one or more algorithms. In some embodiments, one or more processors 104 are configured to execute a trained machine learning model to determine bone density and/or other information as described herein. In some embodiments, processor(s) 104 are configured to cause a machine learning model to be trained using training information. In some embodiments, the machine learning model is trained by providing the training information as input to the machine learning model. In some embodiments, the machine learning model may be and/or include mathematical equations, algorithms, plots, charts, networks (e.g., neural networks), and/or other tools and machine learning model components. For example, the machine learning model may be and/or include one or more neural networks having an input layer, an output layer, and one or more intermediate or hidden layers. In some embodiments, the one or more neural networks may be and/or include deep neural networks (e.g., neural networks that have one or more intermediate or hidden layers between the input and output layers).

As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function that combines the values of all its inputs together. In some embodiments, each connection (or the neural unit itself) may have a threshold function such that a signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free flowing, with connections interacting in a more chaotic and complex fashion.

As described above, the trained neural network may comprise one or more intermediate or hidden layers. The intermediate layers of the trained neural network include one or more convolutional layers, one or more recurrent layers, and/or other layers of the trained neural network. Individual intermediate layers receive information from another layer as input and generate corresponding outputs. In some embodiments, the trained neural network may comprise a deep neural network comprising a stack of convolution neural networks, followed by a stack of long short term memory (LSTM) elements, for example. The convolutional neural network layers may be thought of as filters, and the LSTM layers may be thought of as memory elements that keep track of data history, for example. The deep neural network may be configured such that there are max pooling layers which reduce dimensionality between the convolutional neural network layers. In some embodiments, the deep neural network comprises optional scalar parameters before the LSTM layers. In some embodiments, the deep neural network comprises dense layers, on top of the convolutional layers and recurrent layers. In some embodiments, the deep neural network may comprise additional hyper-parameters, such as dropouts or weight-regularization parameters, for example.

In some embodiments, the trained machine learning model is trained by obtaining and providing prior (e.g., axial slice CT) images to the machine learning model that have been labeled to indicate segmented thoracic and/or lumbar vertebrae, and/or a hip, a spinal column including the lumbar and/or thoracic spine; vertebrae heights; fractured vertebrae and/or other bone; trabecular bone; center(s) of mass of non-fractured vertebrae; a region of interest with only trabecular bone; cortical bone; bone islands; areas with large vessels; calcified herniated discs; a subject's heart tissue, chest mass, and chest wall fat; Schmorl's nodes with sizes, depths, and/or quantities; and corresponding bone density measurements (include mean and/or average densities, etc.), other (e.g., heart, muscle, fat, etc.) tissue density measurements, T scores, Z scores, FRAX scores, and/or other information.

One or more computing devices 106 may be and/or include a smartphone, a laptop computer, a tablet, a desktop computer, a gaming device, and/or other networked computing devices, having a display, a user input device (e.g., buttons, keys, voice recognition, or a single or multi-touch touchscreen), memory (such as a tangible, machine-readable, non-transitory memory), a network interface, an energy source (e.g., a battery), and a processor such as a processor 104 (a term which, as used herein, includes one or more processors) coupled to each of these components. Memory such as electronic storage 138 of computing device 106 may store instructions that when executed by an associated processor provide an operating system and various applications, including a web browser or a native mobile application, for example. In addition, computing device 106 may include a user interface 136, which may include a monitor; a keyboard; a mouse; a touchscreen; etc., User interface 136 may be operative to provide a graphical user interface associated with the system 100 that communicates with imager 102, and/or processor(s) 104, and facilitates user interaction with data from imager 102.

User interface 136 is configured to provide an interface between system 100 and users through which users may provide information to and receive information from system 100. This enables data, results, and/or instructions, and any other communicable items, collectively referred to as “information,” to be communicated between the users and one or more imager 102, processor(s) 104, computing device 106, external resources 108, and/or other components. Examples of interface devices suitable for inclusion in user interface 136 include a keypad, buttons, switches, a keyboard, knobs, levers, a display screen, a touch screen, speakers, a microphone, an indicator light, an audible alarm, a printer, and/or other interface devices. In one embodiment, user interface 136 includes a plurality of separate interfaces (e.g., an interface on imager 102, an interface in computing device 106, etc.). In one embodiment, user interface 136 includes at least one interface that is provided integrally with processor(s) 104. It is to be understood that many communication techniques, either hard-wired or wireless, between one or more components of system 100 are contemplated by the present disclosure. Other exemplary input devices and techniques adapted for use with system 100 as user interface 136 include, but are not limited to, an RS-232 port, RF link, an IR link, modem (telephone, cable or other). In short, any technique for communicating information with system 100 is contemplated by the present disclosure as user interface 136.

Electronic storage 138 comprises electronic storage media that electronically stores information. The electronic storage media of electronic storage 138 may include one or both of system storage that is provided integrally (i.e., substantially non-removable) with system 100 and/or removable storage that is removably connectable to system 100 via, for example, a port (e.g., a USB port, a firewire port, etc.) or a drive (e.g., a disk drive, etc.). Electronic storage 138 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 138 may store software algorithms, information determined by processor(s) 104, information received via user interface 136, information received from imager 102, and/or other information that enables system 100 to function properly. Electronic storage 138 may be (in whole or in part) a separate component within system 100, or electronic storage 138 may be provided (in whole or in part) integrally with one or more other components of system 100 (e.g., computing device 106, processor(s) 104, etc.).

External resources 108, in some embodiments, include sources of information such as databases, websites, etc.; external entities participating with system 100 (e.g., systems or networks associated with system 100), one or more servers outside of the system 100, a network (e.g., the internet), electronic storage, equipment related to Wi-Fi™ technology, equipment related to Bluetooth® technology, data entry devices, or other resources. In some implementations, some or all of the functionality attributed herein to external resources 108 may be provided by resources included in system 100. External resources 108 may be configured to communicate with one or more other components of system 100 via wired and/or wireless connections, via a network (e.g., a local area network and/or the internet), via cellular technology, via Wi-Fi technology, and/or via other resources.

Network 150 may include the internet, a Wi-Fi network, Bluetooth® technology, and/or other wireless technology. In some embodiments, imager 102, one or more processors 104, computing device 106, external resources 108, and/or other components of system 100 communicate via near field communication, Bluetooth, and/or radio frequency; via network 150 (e.g., a network such as a Wi-Fi network, a cellular network, and/or the internet); and/or by other communication methods.

In FIG. 1, imager 102, one or more processors 104, one or more computing devices 106, and/or other components of system 100 are shown as separate entities. This is not intended to be limiting. Some and/or all of the components of system 100 and/or other components may be grouped into one or more singular devices. For example, one or more processors 104 may be included in a computing device 106.

The illustrated components of system 100 are depicted as discrete functional blocks, but embodiments are not limited to systems in which the functionality described herein is organized as illustrated by FIG. 1. The functionality provided by each of the components of system 100 may be provided by software or hardware modules that are differently organized than is presently depicted, for example such software or hardware may be intermingled, broken up, distributed (e.g. within a data center or geographically), or otherwise differently organized. Some or all of the functionality described herein may be provided by one or more processors of one or more computers executing code stored on a tangible, non-transitory, machine readable medium.

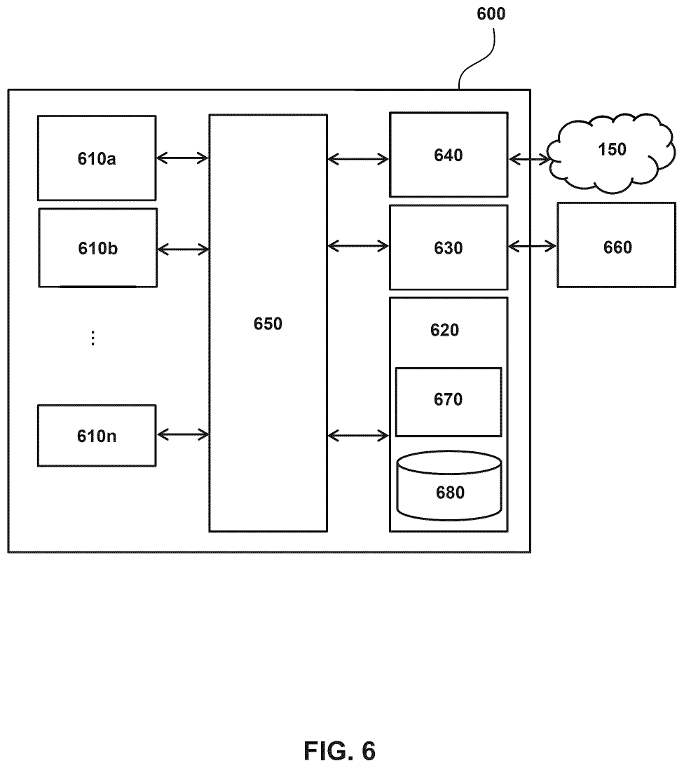

FIG. 6 is a diagram that illustrates an exemplary computing device 600 (similar to and/or the same as computing device 106 described above) in accordance with embodiments of the present system. Various portions of systems and methods described herein, may include, or be executed on one or more computing devices the same as or similar to computing device 600. For example, processor(s) 104 of system 100 (FIG. 1) may be and/or be included in one more computing devices the same as or similar to computing device 600. Further, processes, modules, processor components, and/or other components of system 100 described herein may be executed by one or more processing systems similar to and/or the same as that of computing device 600.

Computing device 600 may include one or more processors (e.g., processors 610a-610n, which may be similar to and or the same as processor(s) 104) coupled to system memory 620 (which may be similar to and/or the same as electronic storage 138, an input/output I/O device interface 630, and a network interface 640 via an input/output (I/O) interface 650. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing device 600. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 620). Computing device 600 may be a uni-processor system including one processor (e.g., processor 610a), or a multi-processor system including any number of suitable processors (e.g., 610a-610n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing device 600 may include a plurality of computing devices (e.g., distributed computer systems) to implement various processing functions.

I/O device interface 630 may provide an interface for connection of one or more I/O devices 660 to computer device 600. I/O devices may include devices that receive input (e.g., from a user) or output information (e.g., to a user). I/O devices 660 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 660 may be connected to computing device 600 through a wired or wireless connection. I/O devices 660 may be connected to computing device 600 from a remote location. I/O devices 660 located on remote computer system, for example, may be connected to computing device 600 via a network and network interface 640.

Network interface 640 may include a network adapter that provides for connection of computing device 600 to a network (e.g., network 150 described above). Network interface 640 may facilitate data exchange between computing device 600 and other devices connected to the network (e.g., network 150 shown in FIG. 1). Network interface 640 may support wired or wireless communication. The network may include an electronic communication network, such as the Internet, a local area network (LAN), a wide area network (WAN), a cellular communications network, or the like.

System memory 620 may be configured to store program instructions 670 (e.g., machine readable instructions) and/or data 680. Program instructions 670 may be executable by a processor (e.g., one or more of processors 610a-610n) to implement one or more embodiments of the present techniques. Instructions 670 may include modules and/or components of computer program instructions for implementing one or more techniques described herein with regard to various processing modules and/or components. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 620 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 620 may include a non-transitory computer readable storage medium that may have program instructions stored thereon that are executable by a computer processor (e.g., one or more of processors 610a-610n) to cause the subject matter and the functional operations described herein. A memory (e.g., system memory 620) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times, e.g., a copy may be created by writing program code to a first-in-first-out buffer in a network interface, where some of the instructions are pushed out of the buffer before other portions of the instructions are written to the buffer, with all of the instructions residing in memory on the buffer, just not all at the same time.

I/O interface 650 may be configured to coordinate I/O traffic between processors 610a-610n, system memory 620, network interface 640, I/O devices 660, and/or other peripheral devices. I/O interface 650 may perform protocol, timing, or other data transformations to convert data signals from one component (e.g., system memory 620) into a format suitable for use by another component (e.g., processors 610a-610n). I/O interface 650 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computing device 600 or multiple computing devices 600 configured to host different portions or instances of embodiments. Multiple computing devices 600 may provide for parallel or sequential processing/execution of one or more portions of the techniques described herein.

Those skilled in the art will appreciate that computing device 600 is merely illustrative and is not intended to limit the scope of the techniques described herein. Computing device 600 may include any combination of devices or software that may perform or otherwise provide for the performance of the techniques described herein. For example, computing device 600 may include or be a combination of a cloud-computing system, a data center, a server rack, a server, a virtual server, a desktop computer, a laptop computer, a tablet computer, a server device, a client device, a mobile telephone, s smartphone, a personal digital assistant (PDA), a mobile audio or video player, a game console, a vehicle-mounted computer, a CT scanner, or a Global Positioning System (GPS), or the like. Computing device 600 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computing device 600 may be transmitted to computing device 600 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present invention may be practiced with other computer system configurations.

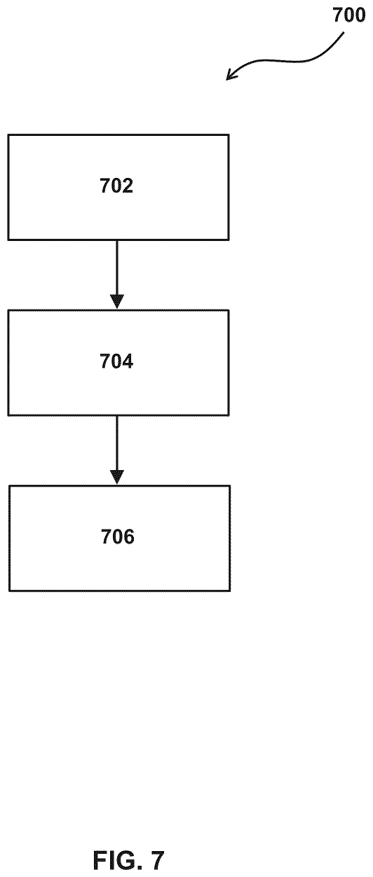

FIG. 7 illustrates a method 700 for determining a bone density measurement. The operations of method 700 presented below are intended to be illustrative. In some embodiments, method 700 may be accomplished with one or more additional operations not described, and/or without one or more of the operations discussed. Additionally, the order in which the operations of method 700 are illustrated in FIG. 7 and described below is not intended to be limiting.

In some embodiments, some or all of method 700 may be implemented in one or more processing devices (e.g., a digital processor, an analog processor, a digital circuit designed to process information, an analog circuit designed to process information, a state machine, and/or other mechanisms for electronically processing information). The one or more processing devices (e.g., processor(s) 104, processor 610a, etc., described herein) may include one or more devices executing some or all of the operations of method 700 in response to instructions stored electronically on an electronic storage medium (e.g., electronic storage 138, system memory 620, etc.). The one or more processing devices may include one or more devices configured through hardware, firmware, and/or software to be specifically designed for execution of one or more of the operations of method 700.

At an operation 702, one or more images of a spine region comprising thoracic and/or lumbar vertebrae, and/or a hip, are obtained by computed tomography (CT). The one or more images may comprise CT images including a lumbar and/or thoracic spine and/or other images. For example, the one or more images may each comprise an axial CT slice. In some embodiments, operation 702 is performed by or with an imager and/or a processor similar to and/or the same as imager 102 and/or processor(s) 104 (shown in FIG. 1 and described herein).

At an operation 704, a spinal column may be identified automatically based on the one or more images. Operation 704 may include automatically detecting thoracic and/or lumbar spinal vertebrae present in the one or more images and segmenting individual vertebrae. A height of each spinal vertebrae may be quantified, and labelled as thoracic or lumbar, for example. In some embodiments, operation 704 includes determining vertebral fractures for any vertebrae with a height loss more than 20% of an average vertebral height. In some embodiments, operation 704 comprises identifying trabecular bone for each non-fractured vertebrae in the one or more images by identifying a center of mass for each non-fractured vertebra in the one or more images. Operation 704 may include defining a region of interest (ROI) to include only trabecular bone in the one or more images. The size of the ROI may be maximized for each axial CT slice, while avoiding cortical bone, bone islands, vertebral fractures, areas with large vessels, and/or calcified herniated discs based upon Hounsfield units (HU) associated with CT. In some embodiments, operation 704 is performed by a processor the same as or similar to processor(s) 104 (shown in FIG. 1 and described herein).

At an operation 706, a bone density measurement is determined based on the one or more images and the identification of the spinal column, and/or other information. Bone from any vertebrae determined to be fractured may be excluded from the bone density measurement. In some embodiments, the bone density measurement comprises a whole body BMD measurement automatically determined by a computing system. In some embodiments, operation 706 includes determining a mean density of the trabecular bone on every available axial CT slice in the one or more images. Operation 706 may include averaging the mean density from each slice to obtain a bone mineral density (BMD) measurement. In some embodiments, operation 706 comprises using a subject's heart density, chest mass, and chest wall fat to calibrate patient, scanner, and scan parameters used to determine the bone density measurement. Calibrating may comprise converting HU associated with CT to Mg/cc of CaHA, for example. In some embodiments, operation 706 comprises determining a T score and a Z score for thoracic, lumbar, both thoracic and lumbar spines, and/or a hip, depending on what is included in the one or more images. In some embodiments, operation 706 comprises determining a Schmorl's node size, depth, and/or quantity. A FRAX (or FRAX-CT) score may be determined based on the Schmorl's node size, depth, and/or quantity; a BMD for the thoracic and/or lumbar spines, and/or a hip; and/or a whole body BMD. In some embodiments, operation 706 is performed by a processor the same as or similar to processor(s) 104 (shown in FIG. 1 and described herein).

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may provided by sending instructions to retrieve that information from a content delivery network.

Various embodiments of the present systems and methods are disclosed in the subsequent list of numbered clauses. In the following, further features, characteristics, and exemplary technical solutions of the present disclosure will be described in terms of clauses that may be optionally claimed in any combination:
1. A method, comprising: obtaining one or more images of a spine region comprising thoracic and/or lumbar vertebrae, and/or a hip, by computed tomography (CT); identifying a spinal column automatically based on the one or more images; and determining a bone density measurement based on the one or more images and an identification of the spinal column.
2. The method of clause 1, wherein the one or more images comprise CT images including a lumbar and/or thoracic spine.
3. The method of any of the previous clauses, further comprising automatically detecting thoracic and/or lumbar spinal vertebrae present in the one or more images and segmenting individual vertebrae.
4. The method of any of the previous clauses, further comprising quantifying anterior and posterior heights of each spinal vertebrae, and labelling each spinal vertebrae as thoracic or lumbar.
5. The method of any of the previous clauses, further comprising determining vertebral fractures for any vertebrae with a height loss more than 20% of an average posterior vertebral height or adjacent non-fractured vertebrae.
6. The method of any of the previous clauses, further comprising excluding bone from any vertebrae determined to be fractured from the bone density measurement.
7. The method of any of the previous clauses, further comprising identifying trabecular bone for each non-fractured vertebrae in the one or more images by identifying a center of mass for each non-fractured vertebra in the one or more images.
8. The method of any of the previous clauses, further comprising defining a region of interest (ROI) to include only trabecular bone in the one or more images.
9. The method of any of the previous clauses, wherein the one or more images each comprise an axial CT slice.
10. The method of any of the previous clauses, further comprising maximizing a size of the ROI for each axial CT slice, while avoiding cortical bone, bone islands, vertebral fractures, areas with large vessels, and/or calcified herniated discs based upon Hounsfield units (HU) associated with CT.
11. The method of any of the previous clauses, further comprising determining a mean density of the trabecular bone on every available axial CT slice in the one or more images.
12. The method of any of the previous clauses, further comprising averaging the mean density from each slice to obtain a bone mineral density (BMD) measurement.
13. The method of any of the previous clauses, further comprising using a subject's heart density, chest/abdominal muscle mass, and/or chest/abdominal/hip fat to calibrate patient, scanner, and scan parameters used to determine the bone density measurement.
14. The method of any of the previous clauses, wherein calibrating comprises converting HU associated with CT to Mg/cc of calcium hydroxyapatite (CaHA).
15. The method of any of the previous clauses, further comprising determining a T score and a Z score for thoracic, lumbar, both thoracic and lumbar spines, and/or a hip, depending on what is included in the one or more images.
16. The method of any of the previous clauses, further comprising determining a Schmorl's node size, depth, location, and/or quantity.
17. The method of any of the previous clauses, further comprising determining a FRAX score based on the Schmorl's node size, depth, location, and/or quantity; a BMD for the thoracic and/or lumbar spines, and/or a hip; and/or a whole body BMD.
18 The method of any of the previous clauses, wherein the bone density measurement comprises a whole body BMD measurement automatically determined by a computing system.

The reader should appreciate that the present application describes several inventions. Rather than separating those inventions into multiple isolated patent applications, applicants have grouped these inventions into a single document because their related subject matter lends itself to economies in the application process. But the distinct advantages and aspects of such inventions should not be conflated. In some cases, embodiments address all of the deficiencies noted herein, but it should be understood that the inventions are independently useful, and some embodiments address only a subset of such problems or offer other, unmentioned benefits that will be apparent to those of skill in the art reviewing the present disclosure. Due to costs constraints, some inventions disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such inventions or all aspects of such inventions.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X′ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device.

What is claimed is:

1. A method, comprising:
obtaining one or more images of a spine region comprising thoracic and/or lumbar vertebrae, and/or a hip, by computed tomography (CT);
identifying a spinal column automatically based on the one or more images; and
determining a bone density measurement based on the one or more images and an identification of the spinal column.

2. The method of claim 1, wherein the one or more images comprise CT images including a lumbar and/or thoracic spine.

3. The method of claim 1, further comprising automatically detecting thoracic and/or lumbar spinal vertebrae present in the one or more images and segmenting individual vertebrae.

4. The method of claim 3, further comprising quantifying a height of each spinal vertebrae, and labelling each spinal vertebrae as thoracic or lumbar.

5. The method of claim 4, further comprising determining vertebral fractures for any vertebrae with a height loss more than 20% of an average vertebral height.

6. The method of claim 5, further comprising excluding bone from any vertebrae determined to be fractured from the bone density measurement.

7. The method of claim 1, further comprising identifying trabecular bone for each non-fractured vertebrae in the one or more images by identifying a center of mass for each non-fractured vertebra in the one or more images.

8. The method of claim 7, further comprising defining a region of interest (ROI) to include only trabecular bone in the one or more images.

9. The method of claim 8, wherein the one or more images each comprise an axial CT slice.

10. The method of claim 9, further comprising maximizing a size of the ROI for each axial CT slice, while avoiding cortical bone, bone islands, vertebral fractures, areas with large vessels, and/or calcified herniated discs based upon Hounsfield units (HU) associated with CT.

11. The method of claim 10, further comprising determining a mean density of the trabecular bone on every available axial CT slice in the one or more images.

12. The method of claim 11, further comprising averaging the mean density from each slice to obtain a bone mineral density (BMD) measurement.

13. The method of claim 1, further comprising using a subject's heart density, chest wall/abdominal and/or hip muscle mass and fat to calibrate patient, scanner, and scan parameters used to determine the bone density measurement.

14. The method of claim 13, wherein calibrating comprises converting HU associated with CT to Mg/cc of calcium hydroxyapatite (CaHA).

15. The method of claim 1, further comprising determining a T score and a Z score for thoracic, lumbar, both thoracic and lumbar spines, and/or a hip, depending on what is included in the one or more images.

16. The method of claim 1, further comprising determining a Schmorl's node size, depth, location, and/or quantity.

17. The method of claim 16, further comprising determining a FRAX-CT score based on the Schmorl's node size, depth, location, and/or quantity; presence of vertebral fractures; measured BMD for the thoracic and/or lumbar spines, and/or a hip; and/or a whole body BMD.

18. The method of claim 1, wherein the bone density measurement comprises a whole body BMD measurement automatically determined by a computing system.
